(19) United States
(12) Patent Application Publication
(10) Pub. No.: US 2015/0223750 A1
(43) Pub. Date: Aug. 13, 2015

(54) METHOD FOR IMPROVING BREATHING PERFORMANCE OF A USER AND AN ELECTRONIC DEVICE THEREFOR

(71) Applicant: Wipro Limited, Bangalore (IN)

(72) Inventors: Raghavendra Hosabettu, Bangalore (IN); Abhijith Vijayakumar Rugmini Bai, Bangalore (IN); Parthasarathy Shunmugam Arumugasamy, Bangalore (IN); Satish Prasad Rath, Bangalore (IN)

(73) Assignee: Wipro Limited, Bangalore (IN)

(21) Appl. No.: 14/228,668

(22) Filed: Mar. 28, 2014

(30) Foreign Application Priority Data
Feb. 12, 2014 (IN) 650/CHE/2014

(51) Int. Cl. A61B 5/00; A61B 5/087; A61B 7/00; A61B 5/097

(52) U.S. Cl. CPC A61B 5/486; A61B 5/097; A61B 5/0878; A61B 7/003; A61B 5/742; A61B 5/7246; A61B 5/7475; A61B 5/7405

(57) ABSTRACT

Embodiments of the present disclosure provide a method of improving breathing performance of a user using an electronic device. In an embodiment, the method of the present disclosure comprises creating a profile of a user by using personal information of the user, determining one or more threshold values corresponding to one or more levels based on the user profile, receiving values associated with at least parameter from respiratory blows of the user, determining breathing performance of the user by comparing the values with a corresponding threshold value, displaying the breathing performance of the user and adjusting the threshold values for successive levels based on the breathing performance.

### RELATED APPLICATIONS

This application claims the benefit of Indian Patent Application Filing No. 650/CHE/2014, filed Feb. 12, 2014, which is hereby incorporated by reference in its entirety.

### FIELD OF THE INVENTION

The present disclosure relates to fitness technology. In particular, embodiments of present disclosure include a method and system for improving breathing performance of a user.

### BACKGROUND OF THE INVENTION

Oxygen is as important for human beings as food and water do. Any nourishment has to be first oxidized to release energy and thereafter the energy can be provided for the body. According to this concern, oxygen is critical for metabolism and probably one of the most important factors for maintaining human's health. Regular metabolism starts from air inhaling and exchanging of the breathing system. A breathing regime usually consists of a repeated sequence of inhalation, exhalation and retention of breath at different intensities, where the relative timing of inhalation and exhalation is important.

People are different in physiological conditions, and there is no absolute criteria that can be determined for a correct breathing pattern. Breathing exercises like yoga and stress relaxation exercises are typically used as part of health and wellness related techniques. In addition, they are also used as supportive measures in the treatment of diseases like asthma and chronic obstructive pulmonary disease (COPD). However, there certainly would be a breathing pattern suitable for a certain people. Proper breathing is a special knowledge, and there are breathing pattern mentors who teach people to breathe properly. Especially, professional instructions for suitable breathing pattern are more needed for those patients suffering serious breathing difficulties. Usually, all of these breathing techniques need to be learned from an instructor/learning instrument and practiced (at home) on a regular basis, preferably daily. Also, following a strict regime of these breathing exercises is dependent on individuals' lifestyle, surroundings and daily schedule. An ordinary breath teaching system providing basic breathing guidance to guide ordinary breath learner to learn anywhere and anytime would be very desirable.

Hence, there exists a need for a system and a method for improving a breathing performance of a user using an electronic device.

### SUMMARY OF THE INVENTION

The shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

Disclosed herein is a method for improving breathing performance of a user using an electronic device. The method comprises determining one or more threshold values corresponding to each of one or more levels for a user based on a profile of the user, receiving one or more values associated with at least one parameter from respiratory blows of the user, determining a breathing performance of the user based on a comparison between each of the one or more values with a corresponding threshold value, and adjusting the one or more threshold values for a successive level based on the breathing performance of the user.

In an aspect of the present disclosure, an electronic device for improving breathing performance of a user is provided. The electronic device comprises at least one processor, an input unit to receive one or more values associated with at least one parameter from respiratory blows of the user and a memory storing instructions executable by the at least one processor. The instructions configure the at least one processor to determine one or more threshold values corresponding to each of one or more levels for a user based on a profile of the user, determine a breathing performance based on a comparison between each of the one or more values with a corresponding threshold value, and adjust the one or more threshold values for a successive level based on the breathing performance of the user. The electronic device also comprises a display unit to display the breathing performance of the user.

In another aspect of the present disclosure, a non-transitory computer readable medium including instructions stored thereon is provided. The instructions when processed by at least one processor cause an electronic device to perform the acts of determining one or more threshold values corresponding to each of one or more levels for a user based on a profile of the user, receiving one or more values associated with at least one parameter from respiratory blows of the user, determining a breathing performance based on a comparison between each of the one or more values with corresponding threshold value, and adjusting the one or more threshold values for a successive level based on the breathing performance of the user so as to act as motivation.

The foregoing summary is illustrative only and is not intended to be in any way limiting. In addition to the illustrative aspects and features described above, further aspects, and features will become apparent by reference to the drawings and the following detailed description.

### BRIEF DESCRIPTION OF THE DRAWINGS

The novel features and characteristic of the disclosure are set forth in the appended claims. The embodiments of the disclosure itself, however, as well as a preferred mode of use, further objectives and advantages thereof, will best be understood by reference to the following detailed description of an illustrative embodiment when read in conjunction with the accompanying drawings. One or more embodiments are now described, by way of example only, with reference to the accompanying drawings.

FIG. 1 illustrates an electronic device in accordance with some exemplary embodiments of the present disclosure;

FIG. 2 illustrates various exemplary means for receiving parameters of respiratory blows in accordance with some embodiments of the present disclosure;

FIG. 3 shows a flowchart illustrating a method of improving breathing performance of a user in accordance with an embodiment of the present disclosure;

FIG. 4 illustrates an environment with inputs for generating profile of a user in accordance with some embodiments of the present disclosure; and

FIG. 5 illustrates an exemplary pictorial representation of displaying breathing performance of a user in accordance with another some embodiments of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION OF THE INVENTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific aspect disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

Henceforth, embodiments of the present disclosure are explained with the help of exemplary diagrams and one or more examples. However, such exemplary diagrams and examples are provided for the illustration purpose for better understanding of the present disclosure and should not be construed as limitation on scope of the present disclosure.

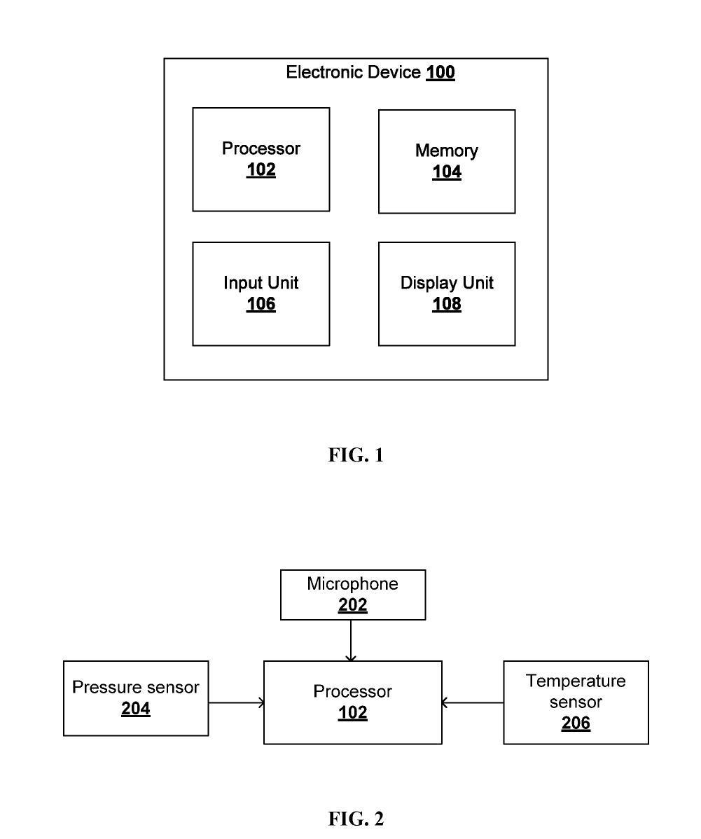

FIG. 1 illustrates an electronic device for improving breathing performance of a user in accordance with some exemplary embodiments of the present disclosure. The electronic device for improving breathing performance of a user may comprise a processor 102, a memory 104, an input unit 106 and a display unit 108. The electronic device 100 for improving user breathing may include, but is not limited to portable terminals, such as a personal digital assistant (PDA), a cellular phone, a personal communication service (PCS) phone, a hand-held PC, a mobile communication terminal, an MP3 player, a notebook computer, a handheld gaming device, a personal media player (PMP), and a navigation terminal.

The input unit 106 may be configured to receive one or more inputs for generating a profile of the user. In some embodiments, the input unit 106 comprises a keypad (not shown in the Fig.) to receive input from user for generating profile of the user. Also, the input unit 106 may be configured to receive one or more values associated with at least one parameter from respiratory blows of the user. In some embodiments, the input unit 106 may capture respiratory blows of the user to receive values for parameters like sound, temperature, pressure and sustenance of the respiratory blows.

The processor 102 may receive the profile of the user and may determine one or more threshold values corresponding to each of one or more levels for a user based on a profile of the user. The processor 102 may further receive the one or more values associated with at least one parameter from respiratory blows of the user. The processor 102 may determine breathing performance of the user by comparing each of the values with corresponding threshold value. Based on the determined breathing performance, the processor 102 may adjust the one or more threshold values for successive level for the user. The processor 102 may comprise a single processor that performs the different functions, or it may comprise multiple processors working together.

The display unit 108 may be configured to display the data concerning the user's breathing to the user. The data concerning user's breathing may include, but is not limited to, breathing performance and one or more levels. In some embodiments, the data concerning the user's breathing may be displayed in at least one of a graphical, a chart or a numerical format. In an embodiment, the display unit 108 may be a touchscreen display. In such an embodiment, the display unit 108 may be additionally configured to receive one or more inputs for generating a profile of the user. The data concerning user's breathing may be also given as an audible signal, or a combination of both display and audio signals. The data concerning user's breathing may be relayed directly to the electronic device 100 of the user whose breath is being monitored or it may be transmitted to a remote display for monitoring by a third party, for example a physiotherapist or a personal trainer.

The memory 104 may be configured to store information including, but not limited to, the profile of the user, one or more initial threshold values for each level for each user and user performance information. In an embodiment, the user performance information may include historical performance of user with respect to time. The memory 104 may be include any of a USB memory of various capacities, a CF memory, an SD memory, a mini SD memory, an XD memory, a memory stick, a memory stick duo, an SMC memory, an MMC memory, and an RS-MMC, for example, noting that alternatives are equally available. Similarly, the memory 104 may be of an internal type included in an inner construction of a corresponding electronic device 100, or an external type disposed remote from such an electronic device 100. Again, the memory 104 may support the above-mentioned memory types as well as any type of memory that is likely to be developed and appear in the near future, such as phase change random access memories (PRAMs), ferroelectric random access memories (FRAMs), and magnetic random access memories (MRAMs), for example.

FIG. 2 illustrates various exemplary means for receiving parameters of respiratory blows in accordance with some embodiments of the present disclosure.

The quality of respiratory blows of each user can be quantified by considering one or more parameters of the respiratory blows. The one or more parameters of the respiratory blows may include, but are not limited to, sound of the respiratory blows, temperature of the respiratory blows, pressure of the respiratory blows and sustenance of the respiratory blows. The processor 102 determines the value of these parameters by receiving input from at least one of microphone 202, a pressure sensor 204 and a temperature sensor 206.

The sound of the respiratory blows is detected by the microphone 202 by detecting an audible aspect of the user's breathing. A person skilled in the art would understand that any device capable of capturing sound of the user can be used instead of microphone 202. The temperature of the respiratory blows is detected by the temperature sensor 206 by detecting an ambient temperature change during an exhale of the user.

The pressure of the respiratory blows is detected by using at least one of a force sensor or pressure sensor 204 of suitable sensitivity. In a non-limiting example, the sensor may be an electret foil or piezoelectric element. Further, the sustenance of the respiratory blows can be determined by a timer, which measures the duration of exhale/inhale of the user.

In an embodiment, the microphone 202, the pressure sensor 204 and the temperature sensor 206 may be integral with the electronic device 102 that incorporates the processor. In an embodiment, the microphone 202, the pressure sensor 204 and the temperature sensor 206 may be connected to the processor by a wired or by a wireless communication.

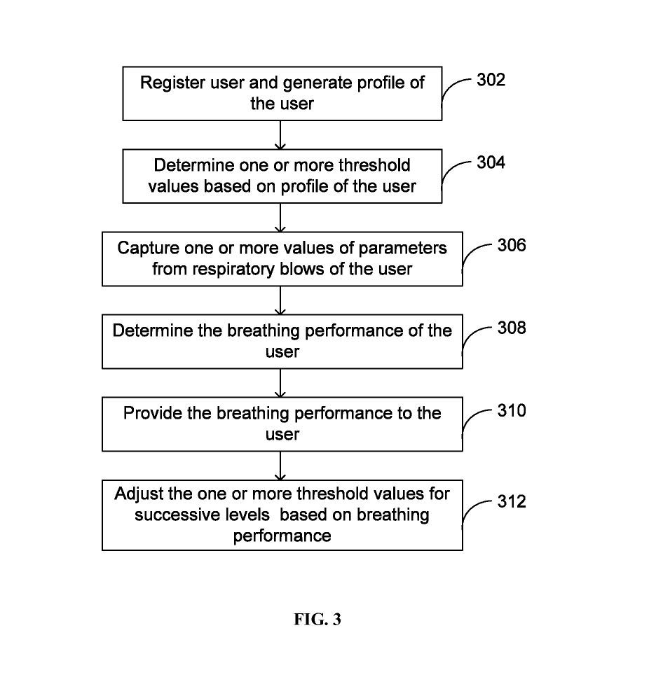

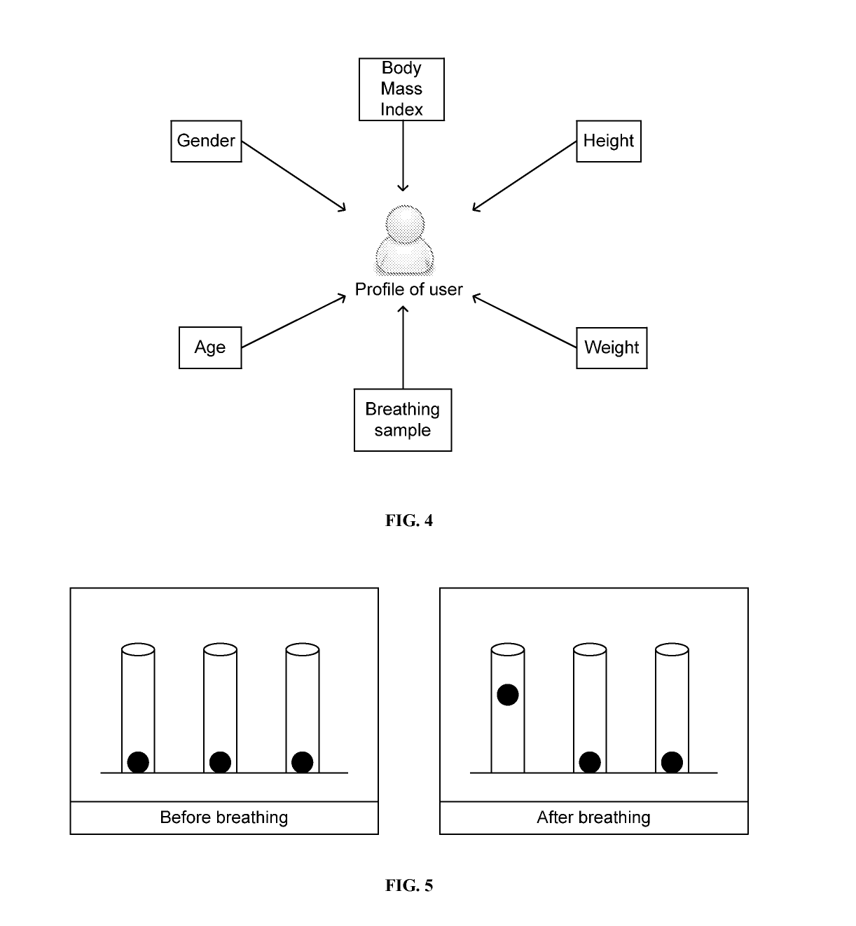

FIG. 3 shows a flowchart illustrating a method of improving breathing performance of a user in accordance with an embodiment of the present disclosure. The user is first registered on an electronic device, for example a mobile phone and a profile of the user is generated simultaneously at step 302. The user provides one or more inputs for generating the profile of the user as illustrated in FIG. 4. The one or more inputs may include, but are not limited to, name, age, gender, height, weight, Body Mass Index (BMI) and breathing sample of the user. The name of the user is input by the user through the keypad of the input unit 106. In some exemplary embodiments, the height of the user may be determined by using an image of the user. The image of the user can be received from an image capturing unit (not shown in Fig.) of the electronic device 100 or from memory 104 of the electronic device 100. The image of the user is then used to determine height of the user based on features of the user. In some exemplary embodiments, the gender of the user may be determined by receiving a finger swipe on display unit 108 of the electronic device 100, for example, a touchscreen display. The weight of the user is calculated based on the age and height of the user. In an embodiment, an ideal weight is determined for the given age and height of the user. Based on the ideal weight, the user may be provided with one or more weight ranges, and the user may be prompted to select the weight range which is relevant to his/her weight. Based on confirmation from user, the weight of user is determined. Below is an example of the weight ranges:

First range—(ideal range−10%) to (ideal weight−5%)

Second range—(ideal range−5%) to (ideal weight+5%)

Third range—(ideal range+5%) to (ideal weight+10%)

A person skilled in the art would understand than any other ranges may be used to determine weight in accordance with the present disclosure.

Further, the BMI of the user is calculated based on the determined height and weight of the user. In an exemplary embodiment, the BMI is calculated by dividing the weight of the user (in kilograms) by the height of the user (in metres). Further, the breathing sample of the user is recorded by the electronic device 100. In an embodiment, at least one of the exhale blows and inhale blows of the user is recorded for breathing sample of the user.

All the inputs defining the profile of the user are stored in the memory 104. Based on all the above inputs of profile of the user, an ideal threshold value is determined for the users at step 304. Then, by using the ideal threshold value, one or more threshold values are determined for the user. The one or more threshold values are stored in the memory 104 used for calculating a breathing performance of the user. As an example, the threshold values of the one or more levels may be calculated as below:

1^{st} level=“>x % of the ideal threshold value”

2^{nd} level=“Ideal threshold value”

3^{rd} level=“<x % of the ideal threshold value”

Where ‘x’ may be any numeric value, eg: 5%, 10% etc.

Once the values for one or more levels are determined, the user breathes into the electronic device 100. Then, one or more values associated with parameters from respiratory blows of the user are received using the electronic device 100 at step 306. The at least one parameter of the respiratory blows may include, but are not limited to, sound, temperature, pressure and sustenance of the respiratory blows of the user. In some embodiments, the parameters of inhale/exhale blows of the user can be captured using a microphone 202, a temperature sensor 206, a pressure sensor 204. The timer (not shown) can be used in addition with the above sensors to determine the sustainability of the respiratory blows of the user.

Then at step 308, the breathing performance of the user is determined. The breathing performance is determined by comparing the one or more values with the corresponding threshold values. In some embodiments, the breathing performance is determined in real-time, such that as the user inhales/exhales, the values of parameters associated with inhaling/exhaling are simultaneously compared with the corresponding threshold value. FIG. 5 illustrates an exemplary pictorial representation of displaying breathing performance of a user in accordance with some embodiments of the present disclosure. In some exemplary embodiment, the one or more tubes representing each level and threshold value of user with respect to the level are displayed on the display unit 108 of the electronic device 100. As the user starts breathing, the ball in the first tube raises up. Once the ball in the first tube reaches the top of the tube, then the ball in the second tube starts rising and so on. Now, to complete a level, the user has to keep breathing till the balls in all the three tubes reach the top or the levels. Therefore, sustenance of the breathing is important for clearing a level. For each level, such one or more tubes are displayed. Once a level is completed, similar one or more tubes are displayed for next levels. However, if the user is unable to complete a level, then the same one or more tubes are displayed.

At step 310, the breathing performance of the user is provided to the user, so that the user is aware of his/her performance. The display can include progress of the user, completion of one or more levels by the user, movement to next level upon completion of a previous level. In some embodiments, each level is displayed in an independent track with the user breathing value marked on each of the tracks. In some embodiments, the breathing performance of the user collected over a period of time may be used to build up a picture of breathing over a set period and the breathing pattern can be analysed to see if it is improving or not over the particular time period. The breathing performance can be provided to the user by displaying the breathing performance in form of text, chart, graph etc. Also, the breathing performance may be provided to the user in form of audio through a speaker of the electronic device.

Then at step 312, the one or more threshold values for a successive level are adjusted based on the performance of the user. In some embodiment, the one or more threshold values may be adjusted based on time taken by the user to complete the one or more levels and number of attempts made by the user to complete the one more levels. In some exemplary embodiments, if the user is taking more time than predefined time for completing a level, then the user is not able to match the one or more threshold values and the threshold values may be adjusted by reducing the threshold value of one or more levels to motivate the user and improve his/her current performance.

Similarly, if the user is taking more number of attempts than a predefined number of attempts, e.g. 5 to clear a level, then the one or more threshold values of the level may be modified by reducing the one or more threshold values of the level to motivate the user.

If the user is not able to complete one of the one or more levels, then there is a need for motivating the user to improve his/her breathing. Therefore, the set threshold values may be reduced by a predetermined ‘x %’ for successive level, where ‘x’ can be any numeric value set by the processor 102. In some embodiments, if the breathing performance of the user is as expected, then the one or more threshold values for levels are kept as is, and not changed. In some embodiments, if the performance of the user is higher than the one or more threshold values of the level, then the threshold values of the one or more levels are increased by a predetermined by ‘x %’ for successive level. This way, the user will not be bored and will keep improving his current breathing performance.

In an embodiment, the method of the present disclosure can be implemented as application on a portable electronic device, such that the user can use this application anywhere and at any time. Rewards may be given for motivating the user based on performance of the user.

The described operations may be implemented as a method, system or article of manufacture using standard programming and/or engineering techniques to produce software, firmware, hardware, or any combination thereof. The described operations may be implemented as code maintained in a “non-transitory computer readable medium”, where a processor may read and execute the code from the computer readable medium. The processor is at least one of a microprocessor or a processor capable of processing and executing the queries. A non-transitory computer readable medium may comprise media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. The non-transitory computer-readable media comprise all computer-readable media except for a transitory. The code implementing the described operations may further be implemented in hardware logic (e.g., an integrated circuit chip, Programmable Gate Array (PGA), Application Specific Integrated Circuit (ASIC), etc.).

Still further, the code implementing the described operations may be implemented in “transmission signals”, where transmission signals may propagate through space or through a transmission media, such as an optical fiber, copper wire, etc. The transmission signals in which the code or logic is encoded may further comprise a wireless signal, satellite transmission, radio waves, infrared signals, Bluetooth, etc. The transmission signals in which the code or logic is encoded is capable of being transmitted by a transmitting station and received by a receiving station, where the code or logic encoded in the transmission signal may be decoded and stored in hardware or a non-transitory computer readable medium at the receiving and transmitting stations or devices. An “article of manufacture” comprises non-transitory computer readable medium, hardware logic, and/or transmission signals in which code may be implemented. A device in which the code implementing the described embodiments of operations is encoded may comprise a computer readable medium or hardware logic. Of course, those skilled in the art will recognize that many modifications may be made to this configuration without departing from the scope of the invention, and that the article of manufacture may comprise suitable information bearing medium known in the art.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise. The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIG. 3 show certain events occurring in a certain order. In alternative embodiments, certain operations may be performed in a different order, modified or removed. Moreover, steps may be added to the above described logic and still conform to the described embodiments. Further, operations described herein may occur sequentially or certain operations may be processed in parallel. Yet further, operations may be performed by a single processor or by distributed processing units.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims hereinafter appended.

Additionally, advantages of present disclosure are illustrated herein.

The present disclosure provides a method for determining breathing performance of a user. The present disclosure enables a user to simply and easily correct their own respiratory state to maintain psychological stability anytime and anywhere, e.g., through such a portable system which he or she can always carry around.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference | |
|---|---|
| Number | Description |
| 100 | Electronic Device |
| 102 | Processor |
| 104 | Memory |
| 106 | Input Unit |
| 108 | Display Unit |
| 202 | Microphone |
| 204 | Pressure Sensor |
| 206 | Temperature Sensor |

What is claimed is:

1. A method for improving breathing performance of a user using an electronic device, comprising:
determining one or more threshold values corresponding to each of one or more levels for a user based on a profile of the user;
receiving one or more values associated with at least one parameter from respiratory blows of the user;
determining a breathing performance of the user based on a comparison between each of the one or more values with a corresponding threshold value; and
adjusting the one or more threshold values for a successive level based on the breathing performance of the user.

2. The method as claimed in claim 1, wherein the profile of the user is generated based on at least one of age of the user, height and weight of the user, gender of the user, Body Mass Index (BMI) of the user and breathing sample of the user.

3. The method as claimed in claim 1, wherein the at least one parameter is selected from at least one of sound, temperature, pressure, and sustenance of the respiratory blows.

4. The method as claimed in claim 1, wherein the breathing performance of the user is based on a time taken by the user to complete the one or more levels and a number of attempts made by the user to complete the one or more levels.

5. The method as claimed in claim 1 further comprising displaying the one or more levels and breathing performance of the user on a display unit of the electronic device.

6. An electronic device for improving breathing performance of a user, the electronic device comprising:
at least one processor;
an input unit to receive one or more values associated with at least one parameter from respiratory blows of the user;
a memory storing instructions executable by the at least one processor, wherein the instructions configure the at least one processor to:
determine one or more threshold values corresponding to each of one or more levels for a user based on a profile of the user;
determine a breathing performance based on a comparison between each of the one or more values with a corresponding threshold value; and
adjust the one or more threshold values for a successive level based on the breathing performance of the user.

7. The electronic device as claimed in claim 6, wherein the profile of the user is generated based on at least one of age of the user, height and weight of the user, gender of the user, Body Mass Index (BMI) of the user and breathing sample of the user.

8. The electronic device as claimed in claim 6, wherein the at least one parameter is selected from at least one of sound, temperature, pressure, and sustenance of the respiratory blows.

9. The electronic device as claimed in claim 6, wherein the input unit is selected from at least one of microphone, temperature sensor and a touchscreen display.

10. The electronic device as claimed in claim 6, further comprising a display unit to display the breathing performance and the one or more levels of the user.

11. A non-transitory computer readable medium including instructions stored thereon that when processed by at least one processor cause an electronic device to perform acts of:
determining one or more threshold values corresponding to each of one or more levels for a user based on a profile of the user;
receiving one or more values associated with at least one parameter from respiratory blows of the user;
determining a breathing performance based on a comparison between each of the one or more values with corresponding threshold value; and
adjusting the one or more threshold values for a successive level based on the breathing performance of the user.

12. The medium as claimed in claim 11, wherein the instructions further cause the at least one processor to perform operations comprising displaying the one or more levels and breathing performance of the user on a display unit of an electronic device.
